(19) United States
(12) Patent Application Publication
(10) Pub. No.: US 2008/0086112 A1
(43) Pub. Date: Apr. 10, 2008

(54) MICRODEVICE FOR DETECTING, ACTIVATING AND DELIVERING MOLECULES

(75) Inventors: Leu-Wei LO, Zhunan Town (TW); Chung-Shi YANG, Zhunan Town (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: National Health Research Institute (an Institution of Taiwan, R.O.C.), Miaoli County (TW)

(21) Appl. No.: 11/869,053

(22) Filed: Oct. 9, 2007

Related U.S. Application Data
(60) Provisional application No. 60/828,465, filed on Oct. 6, 2006.

(51) Int. Cl. A61K 9/00; A61B 5/00

(52) U.S. Cl. 604/891.1; 600/309

(57) ABSTRACT

An implantable microdevice for investigating the efficiency of site-specific drug delivery, as well as real-time on-site evaluation of the endogenous or exogenous compounds as a result of the specific physiological stress/changes is described. The unique arrangement of the implantable microdevice makes it possible to carry out several diagnostic and therapeutic tasks concurrently, with or without additional functional coupling to other components or devices. Also described is a microdevice for in situ applying and/or monitoring a photodynamic therapy in a subject and methods of using the microdevice. A system for in situ delivering, detecting, and/or activating one or more samples in a subject is also described.

### CROSS-REFERENCE TO RELATED APPLICATIONS

This application claims priority to U.S. Provisional Patent Application No. 60/828,465, filed on Oct. 6, 2006, entitled “Implantable Microdevice for Detecting, Activating and Delivering Molecules.”

### BACKGROUND OF THE INVENTION

The present invention generally relates to a microdevice, and more particularly, to an implantable microdevice for in situ and simultaneous detection, activation and/or delivery of a sample in a subject in need thereof.

Microdialysis is a technique initially employed to research the pharmacokinetics of neurotransmitters and opioids. Implantable microdevices have been intensively developed for diagnostic, monitoring, and delivery purposes in living subjects. For example, a microdialysis probe can be implanted into the brain to serve as a minimally invasive method for the sampling and monitoring of extracellular neurochemicals such as the excitatory amino acid glutamate, and to serve as a tool for the delivery of therapeutic drugs. Implantable microdevices are common tool for in vivo investigations of brain disorders, including cerebral ischemia.

Implantable fiber optics can be used for in vivo monitoring of signals, such as the extrinsic and intrinsic fluorescent dyes or markers. For example, implantable fiber optics had been used to monitor the extravasation of pre-administered fluorescent nanospheres due to the increased blood-brain barrier permeability following cerebral ischemia.

### BRIEF SUMMARY OF THE INVENTION

The present invention provides an implantable microdevice for investigating the efficiency of site-specific nanosphere drug delivery, as well as real-time on-site evaluation of the endogenous or exogenous compounds as a result of the specific physiological stress/changes. The unique arrangement of the implantable microdevice which is the subject of the present invention makes it possible to carry out several diagnostic and therapeutic tasks concurrently, with or without additional functional coupling to other components or devices.

One aspect of the invention relates to an implantable microdevice for at least one of in situ delivering, detecting, and activating one or more samples in a subject. The implantable microdevice comprises: a microdialysis probe comprising an input end, an output end, an inner volume and a dialysis membrane, wherein the dialysis membrane encloses and defines the inner volume, the input end and the output end extend to the inner volume, the input end is operatively couplable to a sample transporter for delivering a flow-in sample to the inner volume, and the output end is operatively couplable to an assay system for detecting a biological parameter from a flow-out sample; and an energy conductor coupled to the microdialysis probe for transmitting and receiving at least one of energy and a signal to and from at least one of a sample within the inner volume and a biological tissue surrounding the inner volume, wherein the energy conductor has a first end that is operatively couplable to an energy source, and a second end that is operatively couplable to a signal detection system.

Another aspect of the invention relates to method for in situ delivering, detecting, and/or activating one or more samples in a subject. The method comprises: implanting an implantable microdevice according to embodiments of the present invention into the subject; delivering a flow-in sample to the inner volume of the microdialysis probe via the input end of the microdialysis probe; detecting a biological parameter from a flow-out sample out of the output end of the microdialysis probe; transmitting at least one of input-energy and an input-signal to at least one of a sample within the inner volume and a biological tissue surrounding the inner volume via the energy conductor; and detecting at least one of output-energy and an output-signal from at least one of the sample within the inner volume and the biological tissue surrounding the inner volume.

Another aspect of the invention relates to a microdevice for in situ applying and/or monitoring a photodynamic therapy in a subject. The microdevice comprises a microdialysis probe comprising an input end, an output end, an inner volume and a dialysis membrane, wherein the dialysis membrane encloses and defines the inner volume, the input end and the output end extend to the inner volume, the input end is operatively couplable to a syringe pump for delivering a flow-in sample comprising an photoactivatable compound to the inner volume and a target tissue adjacent to the inner volume, and the output end is operatively couplable to an assay system for detecting a biological parameter from a flow-out sample; and one or more optical fibers coupled to the microdialysis probe for transmitting photon energy to activate the photoactivatable compound in at least one of the inner volume and the target tissue and receiving signals from at least one of the target tissue and the photoactivatable compound, wherein the one or more optical fibers have a first end that is operatively couplable to an energy source, and a second end that is operatively couplable to a signal detection system.

Another aspect of the invention relates to a method for in situ applying and/or monitoring a photodynamic therapy in a subject. The method comprises: implanting a microdevice for photodynamic therapy according to an embodiment of the present invention into the subject; delivering a flow-in sample comprising a photoactivatable compound to the inner volume of the microdialysis probe and a target tissue adjacent to the inner volume via the input end of the microdialysis probe; detecting a biological parameter from the flow-out sample out of the output end of the microdialysis probe; transmitting photon energy to the photoactivatable compound in at least one of the inner volume and the target tissue via one or more optical fibers; and detecting one or more signals from at least one of the target tissue and the photoactivatable compound.

Yet another aspect of the invention relates to a system for in situ delivering, detecting, and/or activating one or more samples in a subject. The system comprises: a microdialysis probe comprising an input end, an output end, an inner volume and a dialysis membrane, wherein the dialysis membrane encloses and defines the inner volume, the input end and the output end extend to the inner volume; an energy conductor coupled to the microdialysis probe for transmitting and receiving at least one of energy and a signal to and from at least one of a sample within the inner volume and a biological tissue surrounding the inner volume; and a signal detection system coupled to the output end of the microdialysis probe and an end of the energy conductor.

Additional aspects and advantages of the invention will be set forth in part in the description which follows, and in part will be apparent from the description, or can be learned by practice of the invention, in view of the present disclosure. The features and advantages of the invention will be realized and attained by the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a schematic diagram illustrating an implantable microdevice according to an embodiment of the present invention;

FIG. 1B is a schematic diagram illustrating an implantable microdevice according to an embodiment of the present invention;

FIG. 2 is a graph showing in vitro fluorescent intensity and glutamate concentration measured simultaneously from an liquid preparation containing various concentrations of both fluorescent nanospheres and glutamate using an implantable microdevice according to an embodiment of the present invention; and

FIG. 3 is a graph showing in vivo fluorescent intensity (indicated by the solid line) and glutamate concentration (indicated by the dotted line) measured simultaneously from cerebral vasculature of a rat using an implantable microdevice according to an embodiment of the present invention.

### DETAILED DESCRIPTION OF THE INVENTION

Unless defined otherwise, all technical and scientific terms used herein have the same meaning as commonly understood by one of ordinary skill in the art to which the invention pertains. Although any methods and materials similar or equivalent to those described herein can be used in the practice of testing of the present invention, the preferred materials and methods are described herein.

As used herein, the article “a” or “an” means one or more than one (that is, at least one) of the grammatical object of the article, unless otherwise made clear in the specific use of the article in only a singular sense.

All publications, patents, and patent applications cited herein, whether above or below, are hereby incorporated by reference in their entirety.

For a better understanding of the present invention, some of the terms used herein are explained in more detail. It is also to be understood that the terminology used herein is for the purpose of describing particular embodiments only, and is not intended to be limiting. In describing and claiming the present invention, the following terminology will be used in accordance with the definitions set out below.

Definitions

A “implantable microdevice” refers to any miniature device that can be implanted into the body of an animal for sample delivery, diagnostic and/or therapeutic purposes. The implantable microdevice can be implanted into the body by any means, such as surgically or by insertion into a natural orifice of the animal. If it is necessary, the implantable microdevice can be removed from the body thereafter, without causing great harm to the health of the animal.

As used herein, an “assay device” includes a variety of bioassay devices, systems or platforms designed for qualitative or quantitative analysis of samples or their derivatives made available to the devices, systems or platforms.

As used herein, “biological tissue” or “tissue” is a collection of interconnected cells that perform a similar function within an organism. As used herein, the biological tissue includes the cells, interstitial fluid and microenvironment surrounding the cells. Examples of the basic types of tissues are epithelium tissues, connective tissues, muscle tissues, and nerve tissues.

As used herein, the terms “biological parameters”, “physiological parameter”, and “biological and physiological information” are used interchangeably to refer to measurable biological or physiological indices, values, readings, recordings, measurements, or signals that can be used to indicate an individual's health state.

As used herein, “detecting”, when used in the context of detecting a biological parameter or detecting energy or a signal, means to obtain qualitative or quantitative analysis of the biological parameter and the energy or signal via any means. “Detecting”, when used in these contexts, may be interchangeable with a term such as “collecting” “sensing”, “measuring”, “processing”, “monitoring”, “displaying”, or “imaging of”, or any combination of two or more of the terms.

An “inner volume” is an exchange region or space defined by a dialysis membrane of the microdialysis probe. The inner volume can contain the samples in exchange with tissue interstitial fluid diffusible from the neighboring biological tissues and their tissue environments.

As used herein, the term “labeling dye or marker” includes color-labeled, fluorescent-labeled or radioactive-labeled molecules, isotopes, free radicals, or their derivatives that bind to an exogenous compound or agent for identification or detection using a signal detection system.

As used herein, the term “photactivation” and grammatical forms thereof refer to a process by which, upon absorption of a quantum of energy corresponding to a photon of light having a given wavelength, a chemical compound is enabled to participate in or undergo a chemical reaction at a reaction rate which is greater than the corresponding reaction rate in the absence of photo activation.

As used herein, the term “sample” includes biological or chemical molecules, compounds or substances available for detection, activation or delivery using the implantable microdevice according to embodiments of the present invention. The “sample” can be originated from within an organism, tissue or cell (such as an endogenous compound). The “sample” can also be present and/or active in an individual organism, tissue or cell but originated or provided from outside of the organism tissue or cell (such as an exogenous compound). The “sample” can contain one or more biological molecules, including, but not limited, to proteins, peptides, amino acids, nucleotides, nucleic acids, lipids, fatty acids, polysaccharides, etc. The “sample” can also contain one or more chemical molecules, including, but not limited to, a pharmacological agent, such as a small molecule compound, or a diagnostic agent, such as an agent, e.g., a nanosphere, carrying a labeling dye or marker. The sample can further contain a molecule that is triggered or activated by energy, such as a pro-drug, a photoactivatable compound, which can be used for treatment and/or diagnostic purposes.

As used herein, a “small molecule compound” refers to a small organic compound having a molecular weight of more than about 30 yet less than about 3000. Such compounds comprise functional chemical groups necessary for structural interactions with polypeptides, nucleic acids, or other biological molecules, and typically include at least an amine, carbonyl, hydroxyl or carboxyl group, preferably at least two of the functional chemical groups, and more preferably at least three of the functional chemical groups. The compounds can comprise a cyclic carbon or heterocyclic structure and/or aromatic or polyaromatic structures substituted with one or more of the above-identified functional groups.

As used herein, the term “sample transporter” refers to a device which can, actively or passively, deliver a sample to a target site, such as the inner volume of the microdialysis probe according to embodiments of the present invention and/or to the biological tissue surrounding the inner volume. In one embodiment of the present invention, the sample transporter delivers the sample using a force, such as mechanical force, electromagnetic field, or electro-osmotic pressure. In another embodiment, the sample transporter delivers the sample passively, such as with a surface tension.

As used herein, the “signal detection system” refers to a system for at least collecting, sensing, measuring, processing, displaying and/or imaging a signal received via the energy conductor or the microdialysis probe according to embodiments of the present invention. The signal detection system can also include the function of interpreting or converting the amount of samples sampled by the microdialysis probe into detectable signals for analysis.

As used herein, the term “subject” encompasses any warm-blooded animal, that has been the object of treatment, observation, diagnosis, or experiment. The term “subject” particularly includes a member of the class Mammalia such as, without limitation, humans and nonhuman primates such as chimpanzees and other apes and monkey species; farm animals such as cattle, sheep pigs, goats, and horses; domestic mammals such as dogs and cats; laboratory animals including rodents such as mice, rats, and guinea pigs, and the like. The term does not denote a particular age or sex. Thus, adult and newborn subjects, as well as fetuses whether male or female, are intended to be covered.

As used herein, the term “in situ” means to examine a phenomenon or a characteristic in place where it occurs. The term “in situ” includes some studies intermediate between in vivo and in vitro, such as examining and/or analyzing cells in a tissue that is separated from an animal body. The term “in situ” also includes some in vivo studies, such as studies via a microdevice according to embodiments of the present invention that is implanted within an animal body.

The terms “exciting”, “activating” and “stimulating” are used interchangeably here to mean a process by which a molecule is triggered with the energy transmitted via the energy conductor to an excited or activated state.

Embodiments of the present invention are directed to an implantable microdevice for in situ detection, activation and/or delivery of one or more samples in a subject in need thereof. The implantable microdevice comprises a microdialysis probe and an energy conductor coupled to the microdialysis probe. The implantable microdevice can be used to simultaneously conduct two or more functions, such as in situ detection, activation and/or delivery of one or more samples. The samples that are detected, activated, and delivered by the implantable microdevice can be same or different.

In one embodiment of the present invention, a sample transporter delivers a flow-in sample to an inner volume of the microdialysis probe via an input end of the microdialysis probe. Examples of the sample transporter include a syringe pump, a dispensing apparatus, or other delivery devices available for transporting the sample to the inner volume. The inner volume is enclosed and defined by a dialysis membrane of the microdialysis probe. One or more compounds in the flow-in sample may diffuse out of the inner volume by the process of dialysis. The compounds may then interact with a biological tissue surrounding the inner volume to assert their functions, e.g., as a diagnostic agent or a therapeutic agent. One or more biological molecules or other molecules in the surrounding biological tissue may also diffuse into the inner volume by the process of dialysis. A sample in the inner volume may contain compounds of the flow-in sample and compounds present in the biological tissue surrounding the inner volume. Thus, a flow-out sample from the inner volume may contain different compounds or molecules as compared to the flow-in sample. The flow-out sample can be detected by an assay system connected to an output end of the microdialysis probe. The assay system can acquire and analyze biological and physiological information from the inner volume and/or biological tissues surrounding the inner volume. A photoactivatable compound can be delivered to a target site, either via the microdialysis probe, i.e., as part of the in-flow sample, or via a method or means independent of the implantable microdevice, e.g., via injection. The target tissue can be at or near the biological tissue surrounding the inner volume of the microdialysis probe.

The energy conductor can be coupled to an energy source for transmitting energy to the inner volume or/and biological tissues in close proximity to the energy conductor. Also, the energy conductor can be coupled to a signal detection system for receiving and detecting signals released from the sample in the inner volume or/and the biological tissues. For example, the energy conductor, e.g., one or more optical fibers, transmits energy, such as photon energy, to activate the photoactivatable compound. The photoactivated compound can be used for diagnostic and/or treatment purposes. Signals from the photoactivatable compound, before and/or after photoactivation, and/or signals from the biological tissues at the target site, can be detected by a signal detection system that is coupled to the energy conductor.

The energy conductor can be an electromagnetic wave conductor, an electron conductor or a combination of both. Electromagnetic wave may include visible light, X-rays or other electromagnetic waves of various frequencies and amplitudes. In a particular embodiment of the present invention, the energy conductor comprises a plurality of optical fibers.

In one embodiment of the invention, the implantable microdevice comprises an outer casing for holding the microdialysis probe and the energy conductor, e.g., one or more optical fibers, together, wherein the outer casing has one or more window openings for exposing the dialysis membrane and the one or more optical fibers. As an example, the outer casing can have a sharp end to penetrate through the skin to reach the target biological tissue of the subject without prior surgical incision. For other examples of the implantable microdevice without a sharp end, a minimally invasive operation can be required to implant the microdevice adjacent to the target biological tissue.

The implantable microdevice can be designed as a portable microdevice and can be coupled to various detection or assay systems or stations available so far for in situ and simultaneously detection, activation and delivery of sample in a subject. Alternatively, the implantable microdevice can be integrated or built within the currently available detection assay system for achieving in situ and simultaneous detection, activation and delivery. It is noted that the arrangements of the energy conductor are not limited to specific configurations described herein. Other configurations of the energy conductor with respect to the microdialysis probe can also be possible as long as the microdialysis probe and the energy conductor are both exposed to the same sampling area. For example, the microdialysis probe can be coupled to the energy conductor that is arranged parallel to the microdialysis probe.

The implantable microdevice can be further equipped with other compact devices, such as a micro-imaging device or mini surgical tool to initiate a surgical procedure in addition to the detecting, activating and delivering functions of the implantable microdevice. In order to protect the microdevice from wear and tear, digestive juices or enzymes inside the biological tissues or lumens, the implantable microdevice can also be covered with other durable or tissue-compatible materials, coatings, tubing, conduits, piping or catheters, as long as the windows are provided to expose the dialysis membrane and the energy conductor.

According to some embodiments of the invention, the energy conductor is an electromagnetic wave conductor, such as one with one or more optical fibers or a plurality of optical fibers arranged in a co-axial array or as a bundle. Therefore, an energy supply that provides an electromagnetic wave such as a laser or lights of selected wavelengths can be coupled to the optical fibers which transmit photon energy to activate the photosensitive or photoactivatable molecules in the inner volume of the dialysis membrane or the biological tissues in proximity to the implantable microdevice. The activated photosensitive molecules can provide the desired in vivo effects. The electromagnetic wave can also provide other functions, such as illumination required for in vivo imaging of the biological tissues, lumens, blood vessels, nerves and cells thereof.

In accordance with other examples of the invention, the energy conductor is an electron conductor, such as one with a single electrode or a plurality of electrodes coupled to a power supply or electrical source. The electrical charges or electrons from the power supply or electrical source can then be transmitted via the electrode to charge or stimulate the sample in the inner volume of the dialysis membrane or the biological tissues in proximity to the implantable microdevice. In one example, the electron conductor can include a conducting electrode and a detection electrode inserted in a muscle tissue of a test animal in the field of electrophysiology. Meanwhile, the microdialysis probe that is coupled to the electron conductor can concurrently supply exogenous compounds and/or record the neurotransmitters or other chemical signals in the same area of the tissue to provide other physiological information of the test animal.

It should be noted that the sample can be present in gaseous, liquid or solid state. In addition, the sample can include exogenous compounds delivered via the microdialysis probe or endogenous compounds that diffuse into the inner volume of the dialysis membrane by a dialysis process. The exogenous compounds can also diffuse out of the dialysis membrane by the dialysis process. The exogenous compounds can include but are not limited to small molecule compounds, pro-drugs, and agents, such as nanospheres, carrying labeling dyes or markers. The dyes or markers can be extrinsic or intrinsic fluorescent, color or radioactive dyes or markers that can be monitored using a signal detection system, such as well-known microscopies, spectrometry, imaging devices and so on.

The implantable microdevice can detect redox reaction derived information, such as blood glucose and oxygen concentration. The microdevice preferably comprises a microdialysis probe, and a plurality of optical fibers arranged adjacent to the microdialysis probe. As the implantable microdevice is inserted into a circulatory system, including but not limited to various vasculature, capillary and blood vessels, the molecules such as oxygen and blood glucose that permeate or diffuse through the dialysis membrane, are pumped out via the microdialysis probe and measured respectively by the corresponding sensors coupled to the output end of the microdialysis probe. Alternatively, sensors can be arranged adjacent to the microdialysis probe, for example at a tip of the microdialysis probe to ensure real-time in vivo measurement of the oxygen and blood glucose level when the microdialysis probe is inserted. The data acquired from the sensors can then be transmitted via a wired or wireless transmission connection to a computer, a data processor station or a display device for manipulating, processing or displaying the data.

A further example of the invention relates to an implantable microdevice for in situ and simultaneous monitoring of changes of at least two biological parameters in a biological tissue. For example, the blood-brain barrier permeability, neurotransmitter change and other biomedical parameters within a brain of a test subject can be measured using the implantable microdevice to provide a correlation in evaluating the test subject's health condition. The microdevice preferably comprises a microdialysis probe, and a plurality of optical fibers arranged substantially adjacent to the microdialysis probe. The microdialysis probe can be coupled to a chromatographic device for determining the level of the neurotransmitter in the brain, the microdialysis probe can also be coupled to a syringe pump for administering fluorescent, colored or radioactive-labeled nanospheres, and the optical fibers can be coupled to an electromagnetic wave to excite fluorescent, colored or radioactive-labeled nanospheres. The optical fibers can be used for in vivo monitoring of the extrinsic and intrinsic fluorescent, colored or radioactive signals released from the nanospheres via connection to a signal detection system, such as various well-known microscopes, spectrometry, imaging devices and so on.

One other example of the invention relates to a microdevice for in situ diagnosing and treating a patient in need of a photodynamic therapy. The microdevice comprises a microdialysis probe comprising an input end, an output end, an inner volume and a dialysis membrane. The dialysis membrane encloses and defines the inner volume. The input end and the output end extend to the inner volume. The input end is operatively couplable to a syringe pump for delivering a flow-in sample comprising exogenous compounds, such as a photoactivatable compound, to the inner volume and a target tissue adjacent to the inner volume. The output end is operatively couplable to an assay system for detecting a biological parameter from a flow-out sample.

The microdevice also comprises one or more optical fibers coupled to the microdialysis probe for transmitting photon energy to activate the exogenous compounds, such as the photoactivatable compound, in at least one of the inner volume and the target tissue and receiving one or more signals from at least one of the target tissue and the photoactivatable compound. The one or more optical fibers have a first end that is operatively couplable to an energy source, and a second end that is operatively couplable to a signal detection system. The microdialysis tube can be surrounded by a plurality of optical fibers which are coupled to a photon energy source to transmit the photon energy to the exogenous compounds. The signals released from the target tissue or/and the exogenous compounds can be received and transmitted via the optical fibers to a biomedical imaging device, where the corresponding image is generated. Alternatively, the plurality of optical fibers can be built within the microdialysis probe for transmitting the photon energy to the target tissue to activate the exogenous compounds and receiving signals from the target tissue or/and the exogenous compounds.

When the microdevice is used to analyze biological and physiological information of the target tissue in a patient prior to the treatment, a variety of bioassay devices coupled to the output end of the microdialysis probe can be used. When the microdevice is used in a photodynamic therapy, the photon energy transmitted via the plurality of optical fibers activates or excites the exogenous compounds including photosensitive or photoactivatable pro-drugs to induce a therapeutically effective treatment for the patient. One or more therapeutic compounds, such as a small molecule compound and a pro-drug can be administered simultaneously or sequentially via the microdialysis probe. In addition, a therapeutic compound that is not photoactivatable can be administered together with a photoactivatable compound in a combined therapy. The microdevice can also be used to evaluate the therapeutic effect of the photodynamic therapy by comparing the biological and physiological information detected before and after the photodynamic therapy, or at various time points, or under various conditions during the treatment. The biological and physiological information can include, for example, cytokine change, blood pressure level, and other parameters of the target tissue. The treatment regime can be adjusted or modified to suit each patient's need based on such evaluation.

The microdevice for in situ diagnosing and treating a patient in need of a photodynamic therapy according to the present invention can be an implantable microdevice, as well as a microdevice that is not implantable. For example, such a microdevice can be formed with or operably attached to an endoscope apparatus and can be inserted into an appropriate lumen for treatment of esophageal cancer, colorectal cancer, etc. Such a microdevice can also be used non-invasively, for example, when used for dermatological diagnosis and treatment.

Other examples of the invention also relate to a system for in situ delivering, detecting, and/or activating one or more samples in a subject. The system comprises a microdialysis probe comprising an input end, an output end, an inner volume and a dialysis membrane. The dialysis membrane encloses and defines the inner volume. The input end and the output end extend to the inner volume. The system also comprises an energy conductor coupled to the microdialysis probe for transmitting and receiving at least one of energy and a signal to and from at least one of a sample within the inner volume and a biological tissue surrounding the inner volume. The system further comprises a signal detection system coupled to the output end of the microdialysis probe and an end of the energy conductor.

In accordance with examples of the invention, the system can further include a sample transporter coupled to the input end of the microdialysis probe for directing a flow-in sample to the inner volume or/and the biological tissues. The system can also include an energy supply coupled to the energy conductor for supplying energy to the sample in the inner volume or/and the biological tissues. As an example of the invention, the energy conductor can be coupled co-axially to the microdialysis probe. The signal detection system can include, but not be limited to, at least one of a bioassay device, a signal acquiring device, a signal processing device, a signal display device and a signal storage device. According to some examples of the invention, the flow-in sample can include exogenous compounds, endogenous compounds or both. Therefore, the system of the invention can also be applicable to in situ diagnosing and treating a patient in need of a photodynamic therapy, in situ and simultaneous monitoring of changes of one or more biological or physiological parameters in a biological tissue, and other in vivo and in vitro laboratory analyses using the implantable microdevice of the invention.

One skilled in the art would understand in view of the present disclosure that the implantable microdevice can be coupled to a variety of delivery devices, energy sources, detectors, sensors, analyzers and other instrumentalities to provide various biomedical applications not limited by the above-described embodiments.

Embodiments of the present invention also relates to a method for in situ delivering, detecting, and/or activating one or more samples in a subject. The method comprises implanting an implantable microdevice according to embodiment of the present invention into the subject; delivering a flow-in sample to the inner volume of the microdialysis probe via the input end of the microdialysis probe; detecting a biological parameter from a flow-out sample out of the output end of the microdialysis probe; transmitting at least one of input-energy and an input-signal to at least one of a sample within the inner volume and the biological tissue surrounding the inner volume via the energy conductor; and detecting at least one of output-energy and an output-signal from at least one of the sample within the inner volume and the biological tissue surrounding the inner volume.

Embodiments of the present invention also relates to a method for in situ applying and/or monitoring a photodynamic therapy in a subject. The method comprises implanting a microdevice for photodynamic therapy according to embodiment of the present invention into the subject; delivering a flow-in sample comprising a photoactivatable compound to the inner volume of the microdialysis probe and the target tissue adjacent to the inner volume via the input end of the microdialysis probe; detecting the biological parameter from a flow-out sample out of the output end of the microdialysis probe; transmitting photon energy to the photoactivatable compound in at least one of the inner volume and the target tissue via one or more optical fibers; and detecting one or more signals from at least one of the target tissue and the photoactivatable compound.

Note that the particular order of the steps in a method according to embodiments of the present invention may vary. The steps involved in a method according to embodiments of the present invention can be performed concurrently or subsequently.

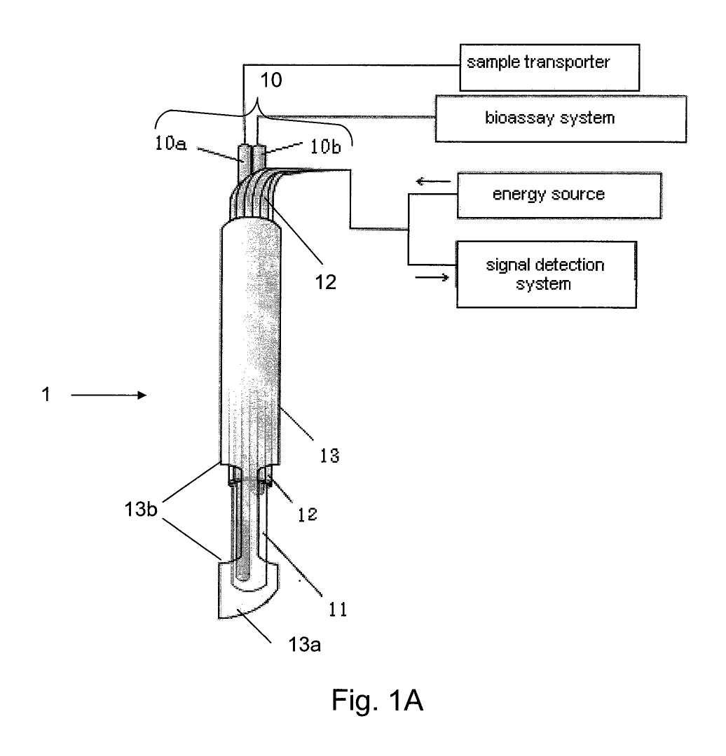

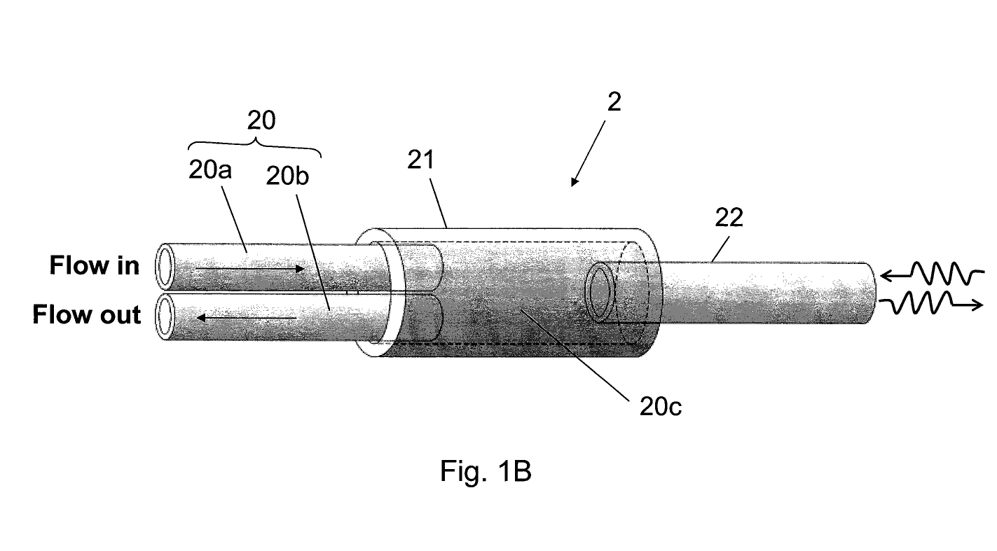

Exemplary implantable microdevices according to embodiments of the present invention are discussed below referring to FIGS. 1A and 1B.

Referring to FIG. 1A, an implantable microdevice 1 comprises a microdialysis probe 10 and an energy conductor 12 that is arranged to surround the microdialysis probe 10. In accordance with one preferred embodiment, the energy conductor 12 comprises a plurality of optical fibers in a co-axial configuration, with each optical fiber having a diameter of about 50 μm. The microdialysis probe 10 has a pair of capillary tubes 10a and 10b of different lengths leading to an inner volume defined by a dialysis membrane 11 of the microdialysis probe 10. The longer capillary tube 10a provides an input end of the microdialysis probe 10 through which the microdialysate enters and is coupled to a sample transporter, such as syringe pump, for delivering the molecules to the inner volume. The shorter capillary tube 10b provides an output end of the microdialysis probe 10 through which the tissue fluid exits and is coupled to a bioassay system, such as chromatography analysis system for acquiring biological and physiological information from the molecules in the inner volume. Both capillary tubes 10a and 10b can be encompassed by the dialysis membrane 11 at their distal ends.

The microdialysis probe 10 and optical fibers 12 can be catheterized into an outer casing 13, e.g., a thin-walled stainless steel tubing having an outer diameter of about 500 μm and an inner diameter of about 350 μm. The stainless steel tubing 13 includes two window openings 13b at both sides of the distal end of the tubing 13. Each window opening has a length of about 4 mm. The windows allow exposure of the dialysis membrane 11 and the optical fibers 12 to the surrounding tissue interstitial fluid. Depending on the tissue microenvironment or interface to be sampled or sensed, one or more additional windows can also be provided on the outer casing 13 to allow direction-specific sensing and sampling of the molecules in the biological tissue.

The outer casing 13 can be shaped to facilitate the implantable microdevice 1 penetrating into a biological tissue without a surgical incision. The outer casing 13 can have a sharp shape 13a at its end for penetration. The outer casing 13 is not limited to any specific shapes or configurations as long as the outer casing 13 provides rigidity, holds the microdialysis probe 10 and energy conductor 12 in the same compartment, and facilitates penetration into the biological tissue. However, embodiments of the present invention also include outer casing 13 that is not shaped to facilitate the implantable microdevice 1 penetrating into a biological tissue without carrying out surgical incision. Invasive operation can be performed to implant such microdevice into the body or tissue of a subject.

Referring to FIG. 1B, an implantable microdevice 2 comprises a microdialysis probe 20 and an energy conductor 22. The microdialysis probe 20 has an input end 20a and an output end 20b, both ends leading to an inner volume 20c, that is defined by a dialysis membrane 21. The input end 20a is used for the delivery of a flow-in sample, and the output end 20b is used for carrying a flow-out sample. The energy conductor 22 is arranged in such a way that it has one end leading to the inner volume 20c, i.e., enclosed within the dialysis membrane 21, like the input end 20a and the output end 20b. This arrangement makes the entire implantable microdevice 2 more compact and easier to be fitted, embedded or implanted inside a biological tissue.

The invention will now be described in further detail with reference to the following specific, non-limiting examples.

### EXAMPLE 1

### In Vitro Measurement of Fluorescent Nanosphere and Glutamate

Series of in vitro assays were performed to characterize an implantable microdevice according to an embodiment of the present invention. The implantable microdevice has the structure of that described in FIG. 1A. The implantable microdevice was placed in a liquid preparation containing either fluorescent nanospheres, glutamate, or both. Fluorescent intensity of fluorescent nanospheres in the liquid preparation was measured via the signal detection system connected to the optical fibers of the implantable microdevice. Glutamate concentration in the liquid preparation was measured from the microdialysate, or the flow-out sample, of the microdialysis probe via a bioassay system connected to the output end of the microdialysis probe. First, measurements of fluorescent intensity and microdialysate were taken individually using liquid preparations containing solely various concentrations of fluorescent nanospheres and liquid preparations containing solely various concentrations of glutamate, respectively. Second, measurements of fluorescent intensity and microdialysate were taken simultaneously using liquid preparations containing various concentrations of both fluorescent nanospheres and glutamate.

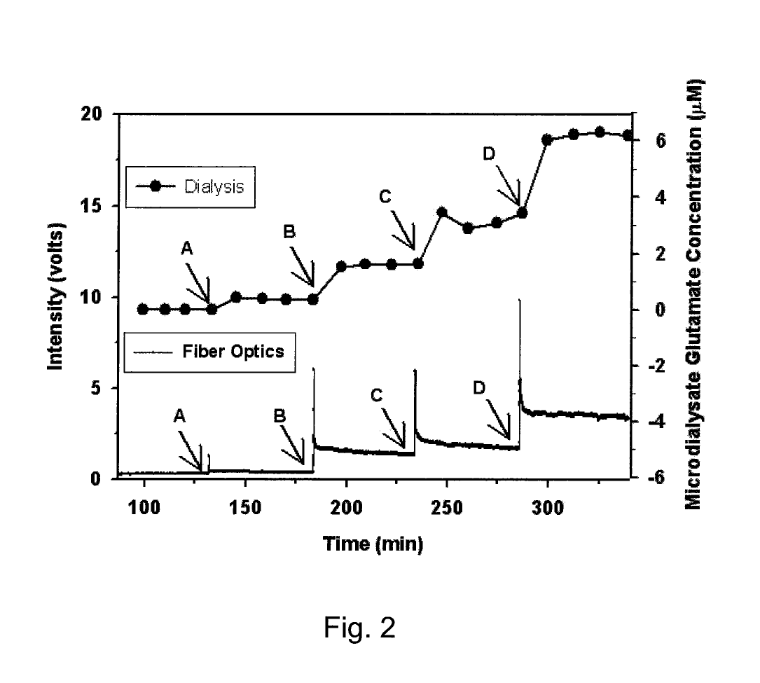

Referring to FIG. 2, a linear dose dependent response was observed when the implantable microdevice was placed in the mixed liquid preparation of fluorescent nanospheres and glutamate. In FIG. 2, A is a time point where the glutamate concentration is about 5 μM and the fluorescent intensity is about 1.625×10¹² nanospheres, B is a time point where the glutamate concentration is about 25 μM and the fluorescent intensity is about 3.25×10¹² nanospheres, C is a time point where the glutamate concentration is about 50 μM and the fluorescent intensity is about 6.5×10¹² nanospheres, and D is a time point where the glutamate concentration is about 100 μM and the fluorescent intensity is about 1.3×10¹³ nanospheres. Therefore, both glutamate concentration and fluorescent intensity were increased over the time.

### EXAMPLE 2

### Monitoring Blood-Brain Barrier Permeability and Neurotransmitter Accumulation Following Cerebral Ischemia

The implantable microdevice of FIG. 1A was also implanted into the cortex of brains of anesthetized rats to monitor the in situ extravasation of pre-administered fluorescent nanospheres (2.6×10¹⁴ nanospheres/ml×1 ml) from the cerebral vasculature, and the level of glutamate following cerebral ischemic insults. The implantable microdevice was then used to simultaneously observe the cerebral ischemia-induced increase in extracellular glutamate concentration, and the increase in blood brain barrier permeability. The extracellular glutamate concentration was measured by microdialysis perfusion and analysis using a chromatographic system coupled to the output end of the microdialysis probe. The blood brain barrier permeability was measured in terms of fluorescent signals released from the extravasated fluorescent nanospheres using the signal detection system coupled to the optical fibers.

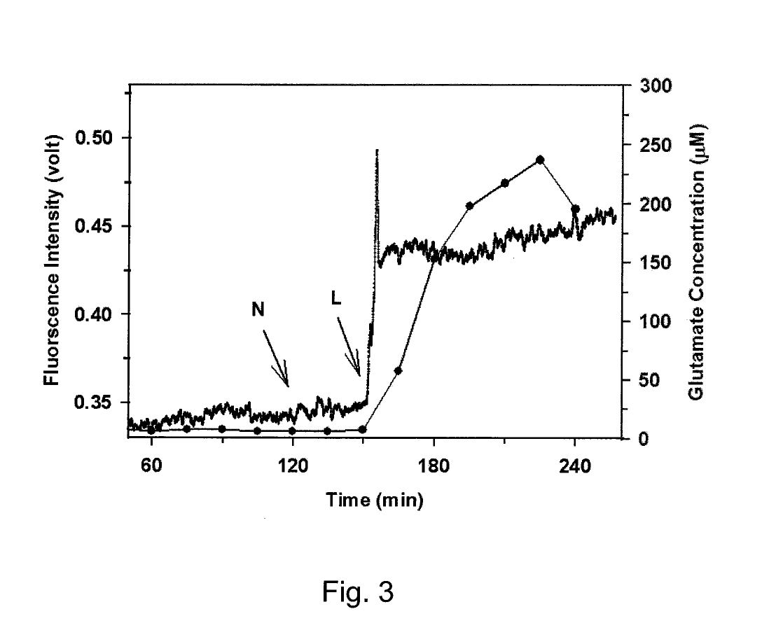

Referring to FIG. 3, N is the time point for injection of nanosphere solution and L is the time point for cerebral ligation. The glutamate concentration and fluorescent intensity remained low following cerebral ischemia. As the cerebral vasculature was ligated, a sharp increase in the glutamate concentration and fluorescent intensity was observed, indicating a cerebral ischemia-induced increase in extracellular glutamate concentration, and an increase in blood brain barrier permeability.

### EXAMPLE 3

### Photodynamic Therapy With Photofrin

Photodynamic therapy has been an effective treatment for patients with certain types of cancer and High-Grade Dysplasia (HGD) associated with Barrett's esophagus, whereby a combination of a photoactivatable drug such as PHOTOFRIN® (Axcan Pharma Inc.) was used. The drug would be absorbed by body tissues, including High-Grade Dysplastic and cancer tissue, and remained in cancer cells, cells with High-Grade Dysplasia associated with Barrett's esophagus, and certain other organs. However, the drug would be largely eliminated from most healthy tissue after a couple of days.

A microdevice according to an embodiment of the present invention can be used in photodynamic therapy. After the photoactivatable drug is injected via the microdialysis probe for approximately 40 to 50 hours, laser light is directed via the optical fibers to the cancer cells or area of High-Grade Dysplasia to activate the drug. Depending on the amount of tumor cells or High-Grade Dysplasia in the treatment, the laser light may be applied for approximately 5 to 40 minutes so as to activate the drug present within those cells and destroy them while limiting damage to surrounding healthy tissue.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. An implantable microdevice for at least one of in situ delivering, detecting, and activating one or more samples in a subject, the implantable microdevice comprising:
a microdialysis probe comprising an input end, an output end, an inner volume and a dialysis membrane, wherein the dialysis membrane encloses and defines the inner volume, the input end and the output end extend to the inner volume, the input end is operatively couplable to a sample transporter for delivering a flow-in sample to the inner volume, and the output end is operatively couplable to an assay system for detecting a biological parameter from a flow-out sample; and
an energy conductor coupled to the microdialysis probe for transmitting and receiving at least one of energy and a signal to and from at least one of a sample within the inner volume and a biological tissue surrounding the inner volume, wherein the energy conductor has a first end that is operatively couplable to an energy source, and a second end that is operatively couplable to a signal detection system.

2. The implantable microdevice according to claim 1, wherein the energy conductor is arranged to surround the microdialysis probe.

3. The implantable microdevice according to claim 2, further comprising an outer casing that holds the microdialysis probe and the energy conductor in the same compartment, wherein the outer casing has at least one window opening for exposing the dialysis membrane and the energy conductor.

4. The implantable microdevice according to claim 3, wherein the outer casing has a sharp end for penetration during implanting the implantable microdevice.

5. The implantable microdevice according to claim 1, wherein the energy conductor extends to the inner volume of the microdialysis probe and is at least partially enclosed within the dialysis membrane of the microdialysis probe.

6. The implantable microdevice according to claim 1, wherein the energy conductor comprises at least one of an electromagnetic wave conductor and an electron conductor.

7. The implantable microdevice according to claim 6, wherein the electromagnetic wave conductor comprises one or more optical fibers, and the electron conductor comprises at least one electrode.

8. The implantable microdevice according to claim 6, wherein the energy conductor comprises a plurality of optical fibers arranged in a co-axial array or bundle.

9. The implantable microdevice according to claim 1, wherein the input end is operatively couplable to a syringe pump for delivering a flow-in sample to the inner volume, the output end is operatively couplable to at least one bioassay device for simultaneously detecting changes of one or more biological parameters from the flow-out sample, and the energy conductor comprises a plurality of optical fibers for transmitting at least one of photon energy and a signal to and from at least one of the sample within the inner volume and the biological tissue surrounding the inner volume.

10. A method for at least one of in situ delivering, detecting, and activating one or more samples in a subject, the method comprising
implanting an implantable microdevice of claim 1 into the subject;
delivering a flow-in sample to the inner volume of the microdialysis probe via the input end of the microdialysis probe;
detecting a biological parameter from a flow-out sample out of the output end of the microdialysis probe;
transmitting at least one of input-energy and an input-signal to at least one of a sample within the inner volume and the biological tissue surrounding the inner volume via the energy conductor; and
detecting at least one of output-energy and an output-signal from at least one of the sample within the inner volume and the biological tissue surrounding the inner volume.

11. The method according to claim 10, further comprising evaluating the health of the subject based on at least one of the biological parameters detected from the flow-out sample and the output-energy and output-signal detected from at least one of the sample within the inner volume and the biological tissue surrounding the inner volume.

12. The method according to claim 10, wherein the flow-in sample comprises at least one of a therapeutic compound and a diagnostic compound, and the flow-out sample comprises at least one of a biological molecule and the diagnostic compound.

13. The method according to claim 10, wherein the flow-in sample comprises a compound selected from the group consisting of a small molecule compound, a pro-drug, and an agent carrying a labeling dye or marker.

14. A microdevice for at least one of in situ applying and monitoring a photodynamic therapy in a subject, the microdevice comprising:
a microdialysis probe comprising an input end, an output end, an inner volume and a dialysis membrane, wherein the dialysis membrane encloses and defines the inner volume, the input end and the output end extend to the inner volume, the input end is operatively couplable to a syringe pump for delivering a flow-in sample comprising an photoactivatable compound to the inner volume and a target tissue adjacent to the inner volume, and the output end is operatively couplable to an assay system for detecting a biological parameter from a flow-out sample; and
one or more optical fibers coupled to the microdialysis probe for transmitting photon energy to activate the photoactivatable compound in at least one of the inner volume and the target tissue and receiving one or more signals from at least one of the target tissue and the photoactivatable compound, wherein the one or more optical fibers have a first end that is operatively couplable to an energy source, and a second end that is operatively couplable to a signal detection system.

15. The microdevice according to claim 14, further comprising an outer casing that holds the microdialysis probe and the one or more optical fibers in the same compartment, wherein the outer casing has at least one window opening for exposing the dialysis membrane and the one or more optical fibers.

16. The microdevice according to claim 14, wherein the one or more optical fibers are arranged to surround the microdialysis probe.

17. The microdevice according to claim 14, wherein each of the one or more optical fibers extends to the inner volume of the microdialysis probe and is at least partially enclosed within the dialysis membrane of the microdialysis probe.

18. The microdevice according to claim 14, wherein the one or more optical fibers include a plurality of optical fibers arranged in a co-axial array or bundle.

19. A method for at least one of in situ applying and/or monitoring a photodynamic therapy in a subject, the method comprising
implanting a microdevice of claim 14 into the subject;
delivering a flow-in sample comprising a photoactivatable compound to the inner volume of the microdialysis probe and a target tissue adjacent to the inner volume via the input end of the microdialysis probe;
detecting a biological parameter from a flow-out sample out of the output end of the microdialysis probe;
transmitting photon energy to the photoactivatable compound in at least one of the inner volume and the target tissue via the one or more optical fibers; and
detecting one or more signals from at least one of the target tissue and the photoactivatable compound.

20. The method of claim 19, wherein the flow-in sample comprises a compound selected from the group consisting of a small molecule compound, a pro-drug, and a nanosphere carrying a labeling dye or marker.

21. The method of claim 19, further comprising evaluating the health of the subject based on a biological parameter detected from a pre-treatment flow-out sample prior to delivering the flow-in sample comprising the photoactivatable compound to the inner volume.

22. The method of claim 19, further comprising evaluating the efficacy of the photodynamic therapy based on at least one of the biological parameter detected from the flow-out sample and the one or more signals detected from at least one of the target tissue and the photoactivatable compound.

23. A system for at least one of in situ delivering, detecting, and/or activating one or more samples in a subject, the system comprising:
a microdialysis probe comprising an input end, an output end, an inner volume and a dialysis membrane, wherein the dialysis membrane encloses and defines the inner volume, the input end and the output end extend to the inner volume;
an energy conductor coupled to the microdialysis probe for transmitting and receiving at least one of energy and a signal to and from at least one of a sample within the inner volume and a biological tissue surrounding the inner volume; and
a signal detection system coupled to the output end of the microdialysis probe and an end of the energy conductor.

24. The system according to claim 23, further comprising a sample transporter coupled to the input end of the microdialysis probe for delivering a flow-in sample to the inner volume.

25. The system according to claim 23, further comprising an energy source coupled to the energy conductor for supplying an energy to at least one of the sample within the inner volume and the biological tissues.
